(19) United States
(12) Patent Application Publication
(10) Pub. No.: US 2008/0045975 A1
(43) Pub. Date: Feb. 21, 2008

(54) Epilation Head and Epilation Device

(75) Inventor: Pedro Sanchez-Martinez, Kronberg (DE)

Correspondence Address: FISH & RICHARDSON PC, P.O. BOX 1022, MINNEAPOLIS, MN, 55440-1022, US

(73) Assignee: BRAUN GMBH, Kronberg (DE)

(21) Appl. No.: 11/576,111

(22) PCT Filed: Jul. 29, 2005
(86) PCT No.: PCT/EP05/08232

§ 371 Date: Apr. 13, 2007

(30) Foreign Application Priority Data
Oct. 1, 2004 (DE) 10 2004 047 874.0

(51) Int. Cl. A61B 17/50; B26B 19/28

(52) U.S. Cl. 606/133; 30/45

(57) ABSTRACT

A motor-driven epilation head for an epilation apparatus. The epilation head includes a rotatable cylinder, which can rotate about an axis of rotation, and several pairs of first clamping elements and second clamping elements that cooperate to take hold of and pluck the hairs. The first clamping elements are individual components that are movably suspended in order to be brought in clamping contact with second clamping elements, and can be individually actuated by means of one respective actuating element. The clamping contact between the first clamping elements and the second clamping elements is disengaged by means of spring elements that act upon the first connecting elements independently of the actuating elements. The axis of rotation of the rotatable cylinder extends outside the first clamping elements.

### TECHNICAL FIELD

The invention pertains to motor-driven epilators and associated epilation heads and clamps.

### BACKGROUND

Epilation apparatuses serve for removing hairs, if possible inclusive of their roots. Known epilation apparatuses are designed, for example, such that the hairs are clamped between adjacent clamping elements and plucked due to a movement of the clamping elements relative to the skin. In this case, the clamping devices need to be closed in a predetermined position in order to take hold of the hairs, then displaced into another predetermined position in the closed state together with the clamped hairs and ultimately opened again in order to release the plucked hairs. This motion sequence can be realized, for example, by arranging the clamping elements on a rotatable cylinder that is set in rotation by means of an electric motor. The opening and the closing of the clamping elements is controlled by means of a control mechanism that may be realized in different ways. The control mechanism typically features actuating elements that act upon the clamping elements such that the clamping elements are respectively closed and opened.

A rotatable cylinder of this type is known, for example, from WO 98/05234 A1. The rotatable cylinder for an epilation apparatus disclosed in this publication is designed such that a separate, individually movable actuating element in the form of a pressing pin is assigned to each pair of clamping elements. The clamping elements are suspended in the rotatable cylinder in such a way that they are closed under the influence of the pressing pins when they act upon the clamping elements on the proximal side of the axis of rotation of the rotatable cylinder referred to the respective clamping surfaces and opened under the influence of the pins when they act upon the clamping elements on the distal side of the axis of rotation.

EP 1 203 544 A1 discloses an epilation apparatus, in which the actuating elements are realized in the form of rods arranged around the axis of the rotatable cylinder. All rods are coupled with a single return spring in such a way that the rods prestress the clamping elements in the direction of the open position. In order to close to clamping elements, the rods are actuated such that the clamping elements are axially displaced against the spring force of the return spring. In the non-actuated state of the rods, they are displaced under the influence of the return spring such that the clamping elements are opened.

JP 2823316 B2 discloses an epilation apparatus with a rotatable cylinder that features stationary and movable clamping elements. The axis of rotation of the rotatable cylinder extends transversely through the clamping elements, such that the movable clamping elements are suspended in the region of the axis of rotation such that they can be tilted relative to the axis of rotation, and thus brought in contact with the stationary clamping elements. The closing movement is generated by means of rods that act upon the movable clamping elements. Spring elements are arranged on the movable clamping elements in order to open the clamping elements.

### SUMMARY

An epilation head for an epilation apparatus is motor-driven and features a rotatable cylinder that can be set in rotation about an axis of rotation. The rotatable cylinder features several pairs of first clamping elements and second clamping elements that cooperate in order to take hold of and pluck the hairs. The first clamping elements are realized in the form of individual components that are movably suspended such that they can be brought in clamping contact with the second clamping elements, such that the first clamping elements can be actuated individually by means of one respective actuating element. The clamping contact between the first and the second clamping elements is disengaged by means of spring elements that are assigned to the first clamping elements and act upon the first clamping elements independently of the actuating elements. The axis of rotation of the rotatable cylinder of the epilation head extends outside the first clamping elements.

The disclosed arrangement of the first clamping elements makes it possible to utilize the available structural space such that numerous pairs of first and second clamping elements can be accommodated in the epilation head. The large number of clamping element pairs makes it possible to pluck hairs almost continuously such that the entire epilation process can be carried out in a relatively painless fashion. A large opening angle can be very quickly reached due to the fact that the spring elements act upon the first clamping elements independently of the actuating elements. This in turn makes it possible to achieve a very thorough epilation. An advantage can be seen in that the first clamping elements that extend no farther than the axis of rotation of the rotatable cylinder are relatively small and therefore have a small mass. This has positive effects on the dynamics of their movements and makes it possible to operate the epilation head with comparatively little noise.

In some embodiments of the epilation head, the spring elements act upon the first clamping elements either directly or by means of machine parts connected to the first clamping elements. In some embodiments, it is preferred to provide one respective spring element per first clamping element. This provides the advantage that the restoring force for each first clamping element is not dependent on the state of the other first clamping elements. Several spring elements may be connected to one another, particularly in an integral fashion, in order to simplify the assembly. In this respect, it is advantageous to connect the spring elements to one another by means of a connecting region that diminishes the effect of the spring elements on one another. The spring elements are realized, for example, in the form of coil springs that are characterized, in particular, by their durability. Machine parts may be provided that are connected to the first clamping elements and accommodate the spring elements in order to simplify the installation of the spring elements, as well as to permanently and securely fix the spring elements. It is also possible to realize the spring elements in the form of leaf springs or wire springs. This results in a multitude of geometric design options.

The second clamping elements may be rigidly arranged in the rotatable cylinder. This simplifies the mechanism and reduces the space requirement. Several second clamping elements are respectively arranged, for example, on a common carrier. In some embodiments, the second clamping elements are arranged axially offset relative to one another over the circumference of the carriers. This makes it possible to realize a gapless plucking region, in which the plucking events take place in rapid succession. The second clamping elements are preferably realized in one piece with the carriers. This makes it possible to maintain the manufacturing and installation expenditures at a low level. The first clamping elements may be movably suspended on the carriers. Blades may be realized, in particular, on the carriers in order to movably suspend the first clamping elements.

The actuating elements are preferably realized in the form of rods that axially impact on the first clamping elements. Such rods can be very inexpensively manufactured and make it possible to realize a simple and robust mechanism for actuating the first clamping elements.

In some embodiments, the first clamping elements are preferably made of metal such that they are able to absorb high mechanical stresses despite their small size, and such that the first clamping elements are also able to reliably clamp the hairs due to their hardness. The second clamping elements are preferably made of a plastic material. This allows a very cost-efficient manufacture. This furthermore makes it possible to maintain a relatively low weight of the epilation head. Another advantage can be seen in the noise and vibration damping achieved during the impact of the first clamping elements.

The disclosure furthermore relates to an epilation apparatus that comprises a hand-held housing and the epilation head.

Other aspects, features, and advantages will be apparent from the following detailed description, the drawings, and the claims.

### DESCRIPTION OF DRAWINGS

The figures show:

FIG. 1 is a side view of an embodiment of an epilation apparatus;

FIG. 2 is a section through the epilation apparatus in the region of the epilation head;

FIG. 3 is a perspective exploded view of the rotatable cylinder;

FIG. 4 is the enlarged detail of FIG. 2 in the region of a clamping element pair;

FIG. 5 is a perspective representation of an embodiment of the spring elements and their arrangement on the carrier disks;

FIG. 6 is a perspective representation of another embodiment of the design and the arrangement of the spring elements;

FIG. 7 is a perspective representation of the design and the arrangement of the spring elements that is modified in comparison with FIG. 6, and

FIG. 8 is a perspective representation of another embodiment of the design and the arrangement of the spring elements.

### DETAILED DESCRIPTION

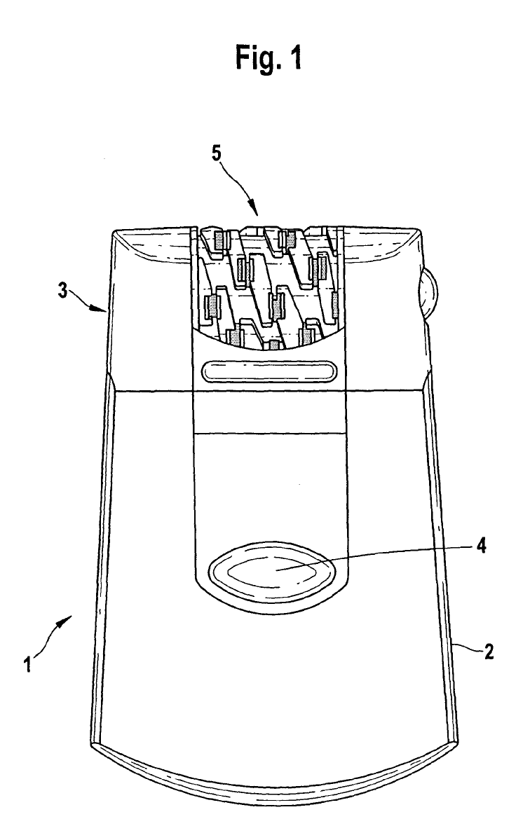

FIG. 1 shows a side view of an embodiment of an epilation apparatus 1. The epilation apparatus 1 includes a housing 2 and an epilation head 3 that is detachably fixed on the housing 2. The housing 2 is shaped such that it can be comfortably held in one hand. A switch 4 for switching the epilation apparatus 1 on and off is arranged on the housing 2. A rotatable cylinder 5 is rotatably suspended in the epilation head 3.

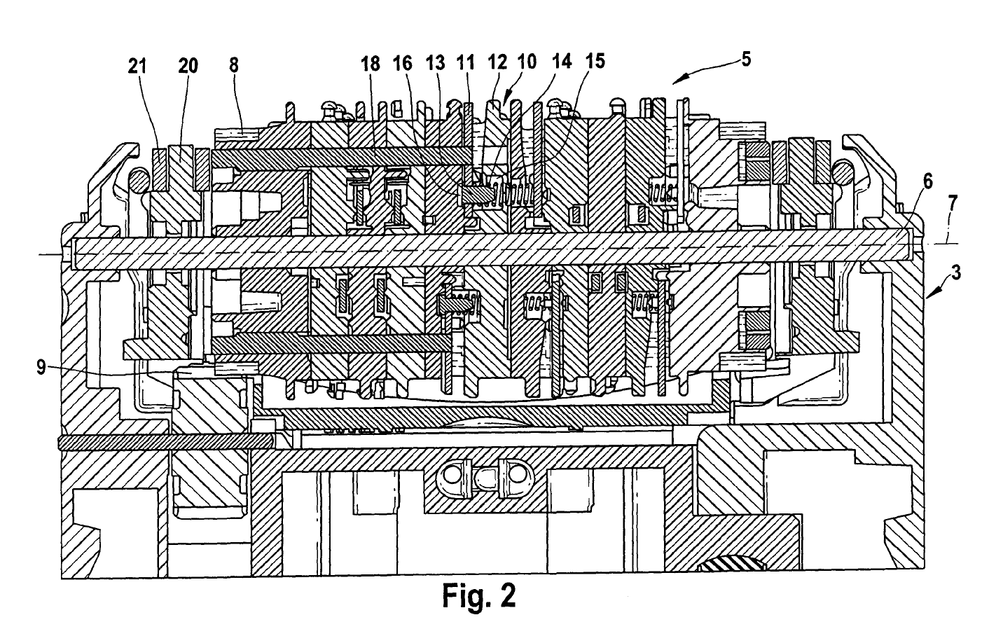

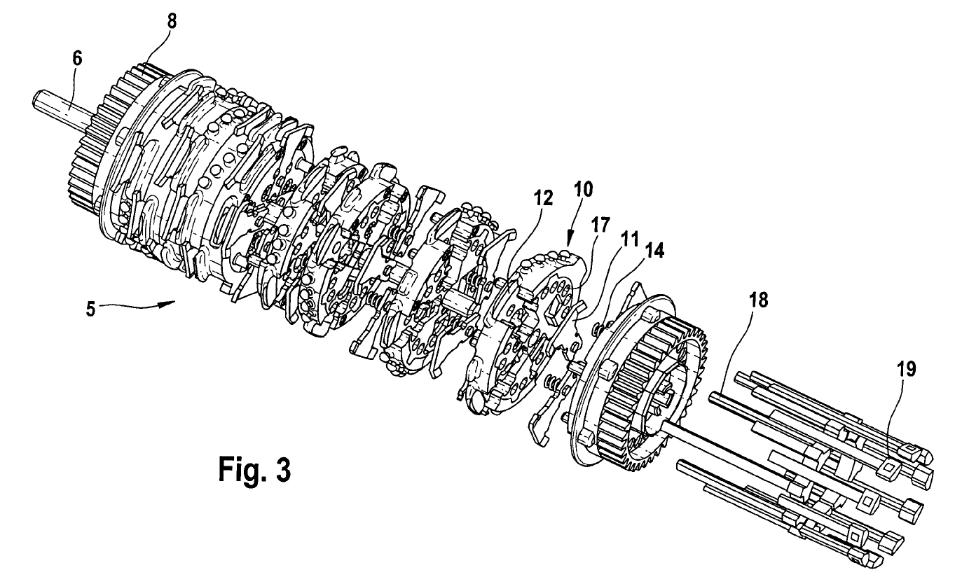

FIG. 2 shows a section through the epilation apparatus 1 in the region of the epilation head 3. A perspective exploded view of the rotatable cylinder 5 is shown in FIG. 3. The rotatable cylinder 5 is rotatably supported in the epilation head 3 by means of a shaft 6 such that the shaft 6 defines an axis of rotation 7 of the rotatable cylinder 5. One respective gear 8 is connected to the rotatable cylinder 5 in a rotationally rigid fashion in the region of the axial ends of the rotatable cylinder 5. One of the gears 8 is engaged with a driving pinion 9 that is driven by an electric motor (not shown) arranged in the interior of the housing 2 via a gear assembly that is also not illustrated in the figures.

In the axial direction, the rotatable cylinder 5 is composed of a multitude of stacked components. The rotatable cylinder 5 features several carrier disks 10. Four movable clamping elements 11 are arranged on each carrier disk 10. In addition, four stationary clamping elements are provided per carrier disk 10, such that the stationary clamping elements are uniformly distributed over the circumference in the region of the outer radius of the carrier disk 10 and slightly offset relative to one another in the axial direction. The stationary clamping elements 12 are realized in one piece with the carrier disk 10 and preferably made of a plastic material. One movable clamping element 11 and one stationary clamping element 12 are respectively arranged axially adjacent to one another. The movable clamping elements 11 are respectively realized in the form of individual components and preferably made of metal. The movable clamping elements 11 extend radially outward beginning at a radius that is larger than the radius of the shaft 6 of the rotatable cylinder 5. Due to their relatively small size in the radial direction, the movable clamping elements 11 have a relatively small mass and therefore can be easily set in motion and stopped again.

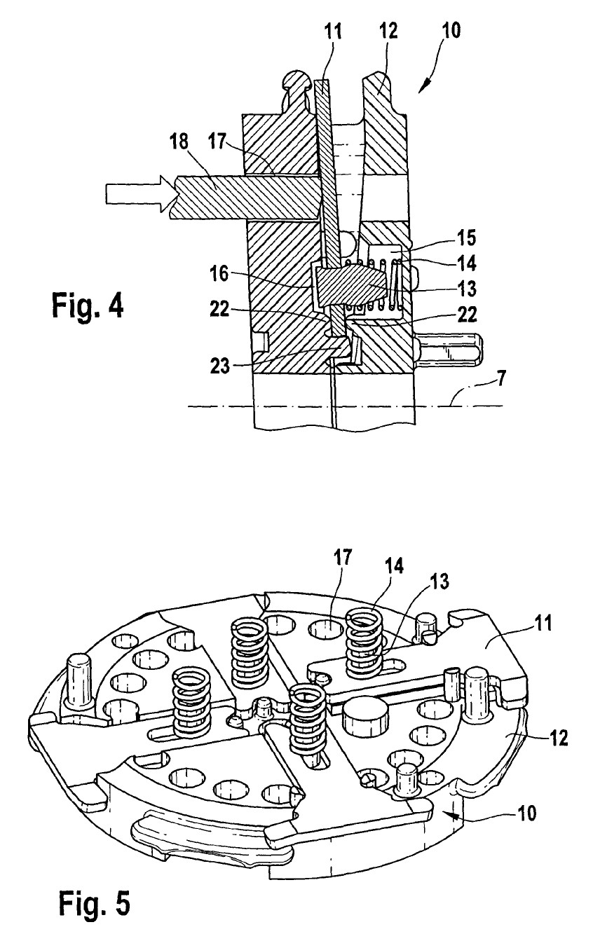

As described in greater detail below with reference to FIG. 4, the movable clamping elements 11 are supported in the vicinity of their inner radial ends such that they can be tilted relative to the axis of rotation 7 of the rotatable cylinder 5, and therefore moved toward and away from the respectively adjacent stationary clamping element 12. This makes it possible to open and close the clamping element pairs that respectively consist of a movable clamping element 11 and a stationary clamping element 12. Each movable clamping element 11 features a pin 13 that is oriented perpendicular to the principal surface of the movable clamping element 11 radially outside its bearing point, such that a spring element 14 in the form of a coil spring is threaded on the aforementioned pin. The pin 13 is sectionally realized in a barrel-shaped fashion in order to fix the spring element 14 on the pin 13. The spring element 14 is supported on the barrel-shaped region of the pin 13 with one end and on the base of a recess 15 in the carrier disk 10 with the other end. The pin 13 completely penetrates the movable clamping element 11 and extends into a depression 16 of the carrier disk 10 on the rear side of the movable clamping element 11, i.e., on the side that faces away from the spring element 14. The spring elements 14 prestress the movable clamping elements 11 such that they move away from the corresponding stationary clamping elements 12. Since separate spring elements 14 are provided, the prestress of each individual movable clamping element 11 is preserved in an unchanged fashion regardless of the condition of the other movable clamping elements 11.

The carrier disks 10 feature several axial through-bores 17 radially outside the recesses 15 or the depressions 16, respectively, such that the through-bores are arranged on a common reference circle and regionally aligned with one another in axially adjacent carrier disks 10. Rods 18 guided in the through-bores 17 partly have a different length. One respective set of rods 18 extends through the through-bores 17 from each axial end of the rotatable cylinder 5 and ends no further than the axial center of the rotatable cylinder 5, such that each rod 18 ends at a different movable clamping element 11 and such that one rod 18 is respectively assigned to each movable clamping element 11. The rods 18 respectively feature a rod head 19 in the region of the axial ends of the rotatable cylinder 5, such that these rod heads are also widened with respect to the rod cross section. One respective roller 21 for actuating the rods 18 is arranged axially adjacent to the rotatable cylinder 5 on a journal 20 on both sides in the region of the graduated circle, on which the through-bores 17 are arranged.

FIG. 4 shows an enlarged detail of FIG. 2 in the region of a clamping element pair. In order to realize the tilting movement of the movable clamping element 11, it is arranged between two blades 22 of the axially adjacent carrier disks 10 in the region of its inner radial end and contacts radially inward an axial projection 23 realized on one of the two adjacent carrier disks 10 in the radially inward direction.

In the operative state of the depilation apparatus 1, the gear 8 of the rotatable cylinder 5 is driven by the driving pinion 9 such that the rotatable cylinder 5 is set in rotation. Due to this rotational movement, the rods 18 with their heads 19 are successively guided past one of the rollers 21. The contact with one of the rollers 21 causes the rods 18 to be respectively displaced in the direction toward the axial center of the rotatable cylinder 5. This means that the rods 18 arranged in the left half of the rotatable cylinder 5 are respectively displaced toward the right, and the rods 18 arranged in the right half of the rotatable cylinder 5 are respectively displaced toward the left. In FIG. 4, the movement of a rod 18 arranged in the left half of the vertical cylinder 5 is indicated with an arrow. The movement of the rod 18 is transmitted to the movable clamping element 11 that is in contact with the rod 18. This results in the movable clamping element 11 being moved toward the adjacent stationary clamping element 12 against the restoring force of the spring element 14 and to ultimately be pressed against this stationary clamping element. This state of the clamping element pairs is respectively reached when they are situated in a position that faces away from the housing 2 of the epilation apparatus 1. If the epilation apparatus 1 is used properly, the clamping element pairs are then respectively situated in the vicinity of the skin surface. A hair is taken hold of if it is located between the movable clamping element 11 and the stationary clamping elements 12 at this time. The clamped hair is plucked out of the skin due to the rotational movement of the rotatable cylinder 5.

The rotational movement of the rotatable cylinder 5 also causes the rod head 19 to disengage from the roller 21 such that the rod 18 is no longer pressed against the movable clamping element 11. Consequently, the movable clamping element 11 is moved away from the adjacent stationary clamping element 12 due to the restoring force of the spring element 14 such that the plucked hair is released. Since this opening movement is generated by the spring element 14 arranged in the region of the movable clamping element 11, a transmission mechanism that originates from the axial ends of the rotatable cylinder 5 is not required for this purpose. The opening movement can be realized without the assistance of the rods 18. The opening movement can be realized very fast without a transmission mechanism, such that it is possible to achieve large aperture angle rapidly, and therefore an efficient depilation. In addition, the required structural space is reduced such that a large quantity of clamping element pairs can be arranged in the rotatable cylinder in order to realize nearly continuous plucking without interruptions. Thorough and painless removal of the hairs can be ensured in this fashion. The arrangement of the movable clamping elements 11 in the radial region outside the shaft 6 also saves structural space and opens up new design options with respect to the arrangement of the movable clamping elements 11, namely because the movable clamping elements 11 of a carrier disk 10 do not overlap in the radial direction.

The epilation apparatus 1 can be modified in many different ways, particularly with respect to the design of the spring elements 14 and, accordingly, the design of the carrier disks 10 in the vicinity of the spring elements 14. This is elucidated below with reference to FIGS. 5 to 8.

FIG. 5 shows an exemplary embodiment of the spring elements 14 and their arrangement on the carrier disks 10 in the form of a perspective representation. In the embodiment shown, the spring elements 14 are once again realized in the form of coil springs. However, the pins 13, on which the spring elements 14 are threaded, are not fixed on the movable clamping elements 11, but rather on the carrier disk 10. In addition, the pins 13 do not have the shape of a barrel, but rather a continuously cylindrical shape. The stationary clamping elements 12 that correspond to the movable clamping elements 11 form components of a carrier disk 10 that would be arranged above the movable clamping elements 11 in the illustration shown in FIG. 5, but was omitted in order to provide a better overview.

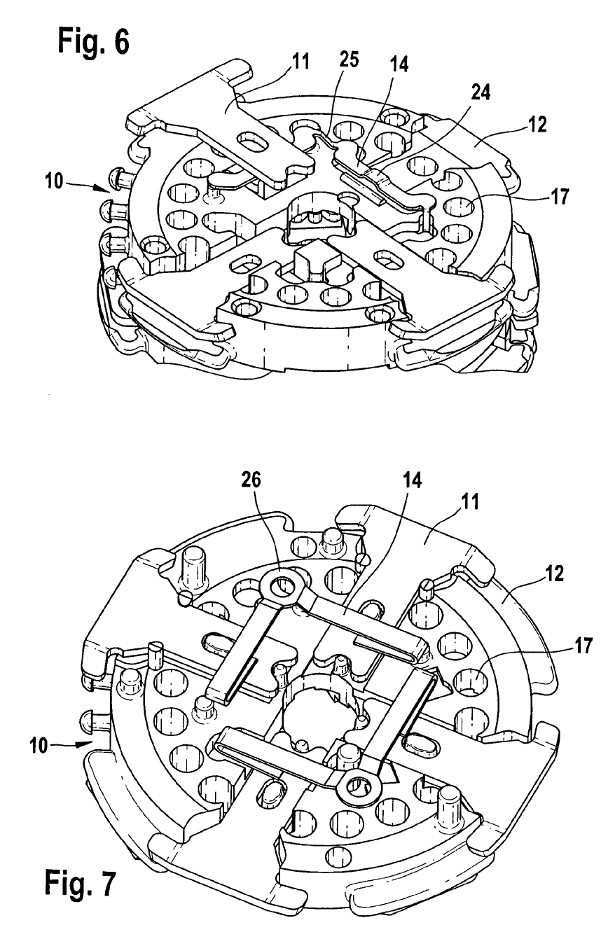

FIG. 6 shows another exemplary embodiment of the design and the arrangement of the spring elements 14 in the form of a perspective representation. The viewing angle is tilted by 180 degrees referred to FIG. 5 such that the spring elements 14 are arranged underneath the movable clamping elements 11. One movable connecting element 11 is removed in order to also provide a view of one of the spring elements 14 in this perspective representation. The spring elements 14 are realized in the form of leaf springs and respectively feature a U-shaped elevation 24, on which one of the movable clamping elements 11 rests. In order to simplify the assembly, two or more spring elements 14 are connected to one another by a bar 25. In order to largely prevent spring elements 14 from influencing one another, the width of bar 25 is very thin such that no noteworthy kinematic coupling between the spring elements 14 is introduced. Instead of bars 25, a ring could also be used that is connected to two or more spring elements 14.

FIG. 7 shows a perspective view of the design and arrangement of the spring elements 14 that is modified in comparison with the embodiment of FIG. 6. In this embodiment, the spring elements 14 once again consist of leaf springs, however, of significantly different shape than the spring elements 14 according to FIG. 6. The spring elements 14 are formed in the shape of a U, such that one respective limb is extended and shaped as one part with a mounting ring 26. In this case, one respective mounting ring 26 is provided for two spring elements 14, i.e., two spring elements 14 are respectively provided per ring 26. The spring elements 14 respectively adjoin one of the movable clamping elements 11 with their shorter limb and are supported on the carrier disk 10 with their longer limb. Alternatively to the drawings shown in FIGS. 6 and 7, leaf springs that are shaped differently may also be considered as spring elements 14.

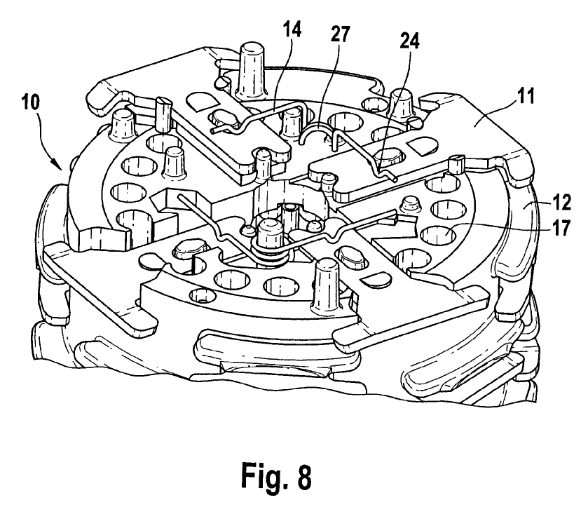

FIG. 8 shows another exemplary embodiment of the design and the arrangement of the spring elements 14 in the form of a perspective representation. In this embodiment, the spring elements 14 are realized in the form of wire springs that have a shape similar to that of the leaf springs shown in FIG. 6. The spring elements 14 according to FIG. 8 respectively feature, in particular, a U-shaped elevation 24, on which one of the movable clamping elements 11 respectively rests. Two respective spring elements 14 are connected to one another by a wire spiral 27 and realized integrally with the wire spiral 27. The wire spiral 27 serves for fixing the spring elements 14 on the carrier disks 10. According to FIG. 8, the wire spiral 27 may be realized in different ways. The two spring elements 14 illustrated in the foreground of FIG. 8 are connected to one another by a wire spiral 27, the axis of which is oriented perpendicular to the principal surface of the carrier disk 10. The two spring elements 14 in the background are connected to one another by a wire spiral 27, the axis of which extends parallel to the principal surface of the carrier disk 10.

Numerous other variations can be realized in addition to the embodiments of the spring elements 14 illustrated in the figures. For example, spring elements 14 that are directed radially inward and realized in the form of leaf springs could be fixed on the movable clamping elements 11 in the region of their radially inner ends, such that said leaf springs then not only serve for the suspension of the movable clamping elements 11, but also for returning these clamping elements in the open position.

1. A motor-driven epilator comprising:
a housing; and
an epilation head mounted on the housing, the epilation head comprising:
a rotatable cylinder that is rotatable about an axis of rotation and having:
several clamping element pairs, each comprising a first clamping element and a second clamping element that cooperate with one another to take hold of and pluck hairs, the first clamping elements each being movably suspended in order to be brought into clamping contact with their associated second clamping elements;
actuator elements that each brings a respective one of the first clamping elements into clamping contact with their associated second clamping elements; and
springs that each act upon a respective one of the first clamping elements independently of the actuating elements to disengage the first clamping element from its associated second clamping element,
wherein the first clamping elements are each disposed completely on a respective side of the axis of rotation.

2. The epilator according to claim 1, wherein the springs act directly upon the first clamping elements.

3. The epilator according to claim 1, wherein one respective spring is provided for each first clamping element.

4. The epilator according to claim 1, wherein several of the springs are connected to one another.

5. The epilator according to claim 4, wherein the springs are connected to one another by a connecting region configured to avoid substantial kinematic coupling.

6. The epilator epilation head according to claim 1, wherein the springs comprise coil springs.

7. The epilator according to claim 6, further comprising machine parts connected to the first clamping elements that accommodate the spring.

8. The epilator according to claim 1, wherein the springs comprise leaf springs.

9. The epilator according to claim 1, wherein the second clamping elements are rigidly arranged in the rotatable cylinder.

10. The epilator according to claim 1, wherein several second clamping elements are respectively arranged on a common carrier.

11. The epilator according to claim 10, wherein the second clamping elements are arranged such that they are axially offset relative to one another over the peripheral surface of the carrier.

12. The epilator according to claim 10, wherein the second elements are formed in one piece with the carrier.

13. The epilator according to claim 10, wherein the first clamping elements are movably suspended on the carrier.

14. The epilator according to claim 13, further comprising blades disposed on the carrier and configured to movably suspend the first clamping elements.

15. The epilator according to claim 1, wherein the actuating elements comprise rods that axially contact the first clamping elements.

16. The epilator according to claim 1, wherein the first clamping elements are made of metal.

17. The epilator according to claim 1, wherein the second clamping elements are made of plastic.

18. The epilation head for an epilator, the epilation head comprising:
a rotatable cylinder that is rotatable about an axis of rotation and having:
several clamping element pairs, each comprising a first clamping element and a second clamping element that cooperate with one another to take hold of and pluck hairs, the first clamping elements each being movably suspended in order to be brought into clamping contact with their associated second clamping elements;
actuator elements that each brings a respective one of the first clamping elements into clamping contact with their associated second clamping elements; and springs that each act upon a respective one of the first clamping elements independently of the actuating elements to disengage the first clamping element from its associated second clamping element,
wherein the axis of rotation of the rotational cylinder extends outside the first clamping elements.

19. The epilator according to claim 4, wherein several springs are connected to one another in an integral fashion.

20. The epilator according to one of claim 1, wherein the springs comprise wire springs.
